(19) United States
(12) Patent Application Publication
(10) Pub. No.: US 2018/0296478 A1
(43) Pub. Date: Oct. 18, 2018

(54) SUBLINGUAL ANTIDEPRESSANT AND ANTIANXIETY TABLET

(71) Applicant: SYNERGISTIC THERAPEUTICS, LLC, Naples, FL (US)

(72) Inventors: Anthony H. Salce, JR., Naples, FL (US); William F. Greenwood, Fairfield, CT (US); Shivsankar Misir, Naples, FL (US)

(73) Assignee: SYNERGISTIC THERAPEUTICS, LLC, Naples, FL (US)

(21) Appl. No.: 16/011,085

(22) Filed: Jun. 18, 2018

Related U.S. Application Data
(63) Continuation-in-part of application No. 15/431,242, filed on Feb. 13, 2017, now Pat. No. 10,034,832.
(60) Provisional application No. 62/294,590, filed on Feb. 12, 2016.

(51) Int. Cl. A61K 9/00; A61K 9/20; A61K 31/135; A61P 25/22; A61P 25/24

(52) U.S. Cl. CPC A61K 9/0056; A61K 9/006; A61P 25/24; A61K 31/135; A61P 25/22; A61K 9/2095

(57) ABSTRACT

Technologies are described for formulations and methods to produce sublingual antidepressant and antianxiety tablets. The tablets may comprise sublingual tablet base and ketamine. The tablets may comprise 2.00 weight percent to 23.00 weight percent ketamine. The methods may comprise placing sublingual tablet base into a chamber. The methods may comprise adding a first ingredient into the chamber. The first ingredient may include ketamine. The methods may comprise mixing the first ingredient into the sublingual tablet base in the chamber to form a mixture. The methods may comprise pressing the mixture into a mold. The methods may comprise drying the mixture in the mold to form the tablet.

### BACKGROUND

Antidepressant and antianxiety treatments may include therapies that target monoaminergic (MA) systems. Antidepressant and antianxiety treatments that target monoaminergic (MA) systems may require 4-6 weeks of administration to achieve effects, may include unpleasant side effects, may possess modest efficacy rates, and may display significant relapse rates. N-Methyl-D-aspartate (NMDA) receptor antagonists may be used as anesthetics and hallucinogenic recreational drugs. Ketamine, diethyl ether, dizocilpine, memantine, phencyclidine, nitrous oxide, and dextromethorphan may be MNDA receptor antagonists.

### SUMMARY

In some examples sublingual antidepressant and antianxiety tablets are described. The tablets may comprise sublingual tablet base and ketamine. The tablets may comprise 2.00 weight percent to 23.00 weight percent ketamine.

In some examples, methods to produce sublingual antidepressant and antianxiety tablets are described. The methods may comprise placing sublingual tablet base into a chamber. The methods may comprise adding a first ingredient into the chamber. The first ingredient may include ketamine. The methods may comprise mixing the first ingredient into the sublingual tablet base in the chamber to form a mixture. The methods may comprise pressing the mixture into a mold. The methods may comprise drying the mixture in the mold to form the tablet.

In some examples sublingual antidepressant and antianxiety tablets are described. The tablets may comprise sublingual tablet base and ketamine. The tablet may comprise 0.005 to 0.050 grams of ketamine.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example system that can be utilized to produce a sublingual antidepressant and antianxiety lozenge;

FIG. 2 illustrates a flow diagram of an example process to produce a sublingual antidepressant and antianxiety lozenge;

FIG. 3 illustrates an example system that can be utilized to produce a sublingual antidepressant and antianxiety tablet;

FIG. 4 illustrates a flow diagram of an example process to produce a sublingual antidepressant and antianxiety tablet;

all arranged according to at least some embodiments described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

It will be understood that any compound, material or substance which is expressly or implicitly disclosed in the specification and/or recited in a claim as belonging to a group or structurally, compositionally and/or functionally related compounds, materials or substances, includes individual representatives of the group and all combinations thereof.

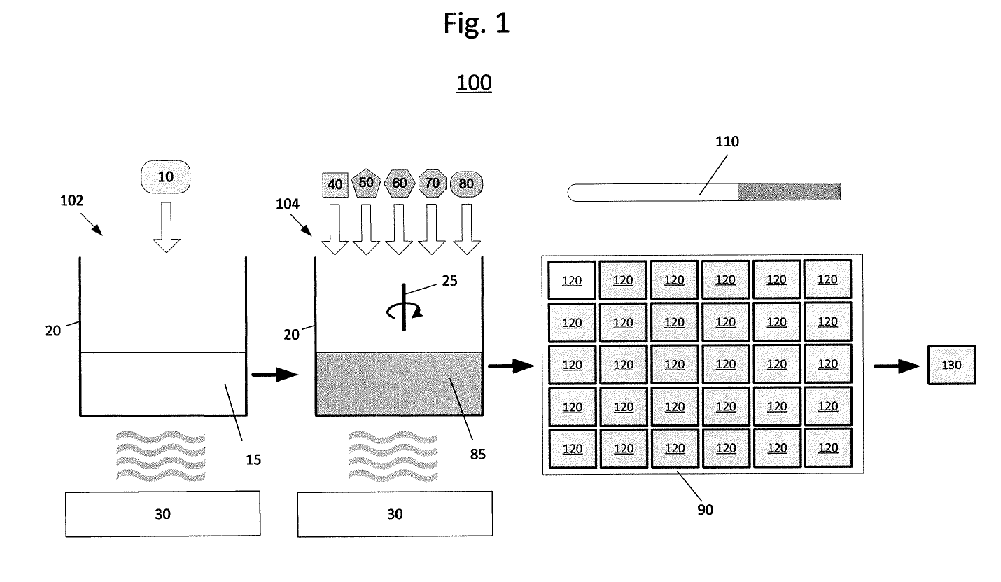

FIG. 1 illustrates an example system that can be utilized to produce a sublingual antidepressant and antianxiety lozenge, arranged in accordance with at least some embodiments presented herein. As discussed in more detail below, a sublingual antidepressant and antianxiety lozenge may be effective in the treatment of depression and anxiety.

System 100 may include a chamber 20, a heater 30 and a lozenge mold 90. At 102 a troche base 10 may be placed in within chamber 20 and melted by heat from heater 30 to produce melted troche base 15. Troche base 10 may be a blend of polyethylene glycols (PEGs). Troche base 10 may be white and/or translucent in appearance and be in the shape of small pellet pieces. Troche base 10 may be solid at room temperatures of 20 to 25 degrees Celsius. Heater 30 may supply heat to increase a temperature of troche base 10 to about 45 to 60 degrees Celsius and melt troche base 10 to produce melted troche base 15.

At 104, a mixing instrument 25 may be inserted into chamber 20 and ingredients 40, 50, 60 70, and 80 may each be individually and respectively added and blended into melted troche base 15. Mixing instrument 25 may be a manual mixing instrument such as a spoon or whisk, or an automated mixer.

Ingredient 40 may be in powder form. Ingredient 40 may include ketamine. Ingredient 40 may include ketamine hydrochloride (HCl) powder.

Ingredient 50 may be in powder form. Ingredient 50 may include silica gel powder. Ingredient 50 may be granular, vitreous in appearance, and porous. Ingredient 50 may be tough and hard in texture. Ingredient 50 may include a strong affinity for water molecules. Ingredient 50 may be silicon dioxide produced synthetically from sodium silicate. Ingredient 50 may have an average pore size of about 2.4 nanometers. Ingredient 50 may be a suspending agent and may keep materials from settling at the bottom of a mold cavity during cooling.

Ingredient 60 may be in powder form. Ingredient 60 may include a weak organic tribasic acid. Ingredient 60 may include citrate. Ingredient 60 may include citric acid powder. Ingredient 60 may include an acidifier, a flavoring, a chelating agent, or a pH adjusting agent. Ingredient 60 may include a processing aid.

Ingredient 70 may be in powder form. Ingredient 70 may include acacia powder. Ingredient 70 may include gum exuded from the acacia tree. Ingredient 70 may include dietary fiber that can dissolve in water. Ingredient 70 may add texture and smoothness to a sublingual antidepressant and antianxiety lozenge.

Ingredient 80 may be in liquid form. Ingredient 80 may be a liquid flavoring. Ingredient 80 may include a liquid confection product. Ingredient 80 may enhance digestion and taste of a sublingual antidepressant and antianxiety lozenge.

As shown at 104, ingredient 40 may be added to chamber 20 and blended into melted troche base 15. Ingredient 40 may be geometrically diluted into melted troche base 15. Ingredient 40 may be mixed until ingredient 40 is evenly distributed throughout melted troche base 15 as indicated by an even distribution of a color of ingredient 40 throughout melted troche base 15.

As shown at 104, ingredient 50 may be added to chamber 20 and blended into melted troche base 15. Ingredient 50 may be geometrically diluted into melted troche base 15. Ingredient 50 may be mixed until ingredient 50 is evenly distributed throughout melted troche base 15 as indicated by an even distribution of a color of ingredient 50 throughout melted troche base 15.

As shown at 104, ingredient 60 may be added to chamber 20 and blended into melted troche base 15. Ingredient 60 may be geometrically diluted into melted troche base 15. Ingredient 60 may be mixed until ingredient 60 is evenly distributed throughout melted troche base 15 as indicated by an even distribution of a color of ingredient 60 throughout melted troche base 15.

As shown at 104, ingredient 70 may be added to chamber 20 and blended into melted troche base 15. Ingredient 70 may be geometrically diluted into melted troche base 15. Ingredient 70 may be mixed until ingredient 70 is evenly distributed throughout melted troche base 15 as indicated by an even distribution of a color of ingredient 70 throughout melted troche base 15.

As shown at 104, ingredient 80 may be added to chamber 20 and blended into melted troche base 15. Ingredient 80 may be mixed until ingredient 80 is evenly distributed throughout melted troche base 15 as indicated by an even distribution of a color of ingredient 80 throughout melted troche base 15.

A melted lozenge mixture 85 may be formed by mixing ingredients 40, 50, 60 70, and 80 into melted troche base 15. Melted lozenge mixture 85 may be poured into cavities 120 of lozenge mold 90. Lozenge mold 90 may be plastic, anodized aluminum, or some other non-permeable material, and may be configured to form equal sized lozenges. Lozenge mold 90 may include 30 uniformly sized cavities 120. Melted lozenge mixture 85 may be poured into cavities 120 of lozenge mold 90 so as to completely fill cavities 120. A scrapper or spatula 110 may be used to level and even out poured melted lozenge mixture 85 in cavities 120 of lozenge mold 90. Spatula 110 may also be used to wipe any excess melted lozenge mixture 85 off of lozenge mold 90.

Lozenge mold 90, with cavities 120 filled with melted lozenge mixture 85, may be cooled to room temperature of 20 to 25 degrees Celsius to form lozenge 130. Lozenge 130 may be a solid lozenge with ingredients 40, 50, 60, 70, and 80 distributed evenly throughout lozenge 130. Lozenge 130 may include about 0.35 weight percent to about 0.65 weight percent of ingredient 40. Lozenge 130 may include about 1.05 weight percent to about 1.35 weight percent of ingredient 50. Lozenge 130 may include about 1.20 weight percent to about 1.55 weight percent of ingredient 60. Lozenge 130 may include about 1.80 weight percent to about 2.10 weight percent of ingredient 70.

### Example 1

Lozenge 130 may include:
0.5 weight percent of ingredient 40;
1.2 weight percent of ingredient 50;
1.38 weight percent of ingredient 60; and
1.98 weight percent of ingredient 70.

### Example 2

A mold with 30 uniformly sized cavities may be utilized to mold 30 lozenges 130 from melted lozenge mixture 85 formed from the following quantities:
0.150 grams of ingredient 40. Ingredient 40 may be ketamine HCl powder.
0.360 grams of ingredient 50. Ingredient 50 may be silica gel powder.
0.414 grams of ingredient 60. Ingredient 60 may be citric acid powder.
0.594 grams of ingredient 70. Ingredient 70 may be acacia powder.
29.850 grams of troche base 10.
6.000 ml of ingredient 80. Ingredient 80 may be tutti frutti flavor liquid.

### Example 3

Lozenge 130 may be formed from the following quantities:
0.005 grams of ingredient 40. Ingredient 40 may be ketamine HCI powder.
0.012 grams of ingredient 50. Ingredient 50 may be silica gel powder.
0.0138 grams of ingredient 60. Ingredient 60 may be citric acid powder.
0.0198 grams of ingredient 70. Ingredient 70 may be acacia powder.
0.995 grams of troche base 10.
0.200 ml of ingredient 80. Ingredient 80 may be tutti frutti flavor liquid.

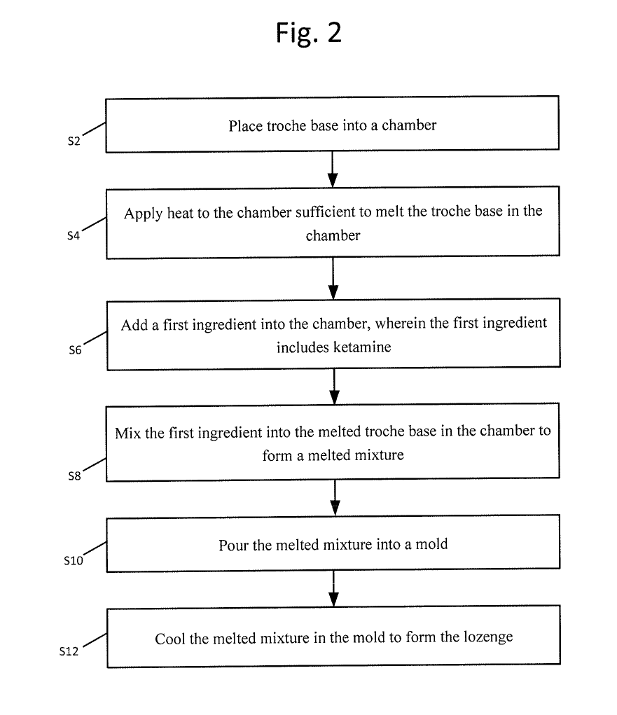

FIG. 2 illustrates a flow diagram of an example process to produce a sublingual antidepressant and antianxiety lozenge 130. The process in FIG. 2 could be implemented using, for example, system 100 discussed above. An example process may include one or more operations, actions, or functions as illustrated by one or more of blocks S2, S4, S6, S8, and/or S10. Although illustrated as discrete blocks, various blocks may be divided into additional blocks, combined into fewer blocks, or eliminated, depending on the desired implementation.

Processing may begin at block S2, “Place troche base into a chamber.” At block S2, a troche base may be placed into a chamber. The troche base may be a blend of polyethylene glycols (PEGs). The troche base may be white and/or translucent in appearance and be in the shape of small pellet pieces. The troche base may be solid at room temperatures of 20 to 25 degrees Celsius.

Processing may continue from block S2 to block S4, “Apply heat to the chamber sufficient to melt the troche base in the chamber.” At block S4, heat may be applied to the chamber sufficient to melt the troche base. Heat may be applied to the chamber sufficient to increase a temperature of the troche base to about 45 to 60 degrees Celsius and melt the troche base.

Processing may continue from block S4 to block S6, “Add a first ingredient into the chamber, wherein the first ingredient includes ketamine.” At block S6, a first ingredient may be added to the chamber. The first ingredient may include ketamine. The first ingredient may include ketamine hydrochloride (HCl) powder.

Processing may continue from block S6 to block S8, “Mix the first ingredient into the melted troche base in the chamber to form a melted mixture.” At block S8, the first ingredient may be mixed into the melted troche base in the chamber. The mixing may be performed by a manual mixing instrument such as a spoon or whisk, or an automated mixer. The first ingredient may be mixed until the first ingredient is evenly distributed throughout the melted troche base as indicated by an even distribution of a color of the first ingredient throughout the melted troche base.

Processing may continue from block S8 to block S10, “Pour the melted mixture into a mold.” At block S10, the melted mixture may be poured into a mold. The mold may be plastic, anodized aluminum, or some other non-permeable material. The mold may be configured with cavities to form uniform sized lozenges. The melted mixture may be poured into the cavities of the mold. The melted mixture may be poured into the cavities of the mold so as to completely fill the cavities of the mold. A scrapper or spatula may be used to level and even out poured melted mixture in the cavities of the mold. The spatula may also be used to wipe any excess melted mixture off of the mold.

Processing may continue from block S10 to block S12, “Cool the melted mixture in the mold to form the lozenge.” At block S12, the melted mixture in the mold may be cooled to form the lozenge. The melted mixture may be cooled to room temperature of 20 to 25 degrees Celsius.

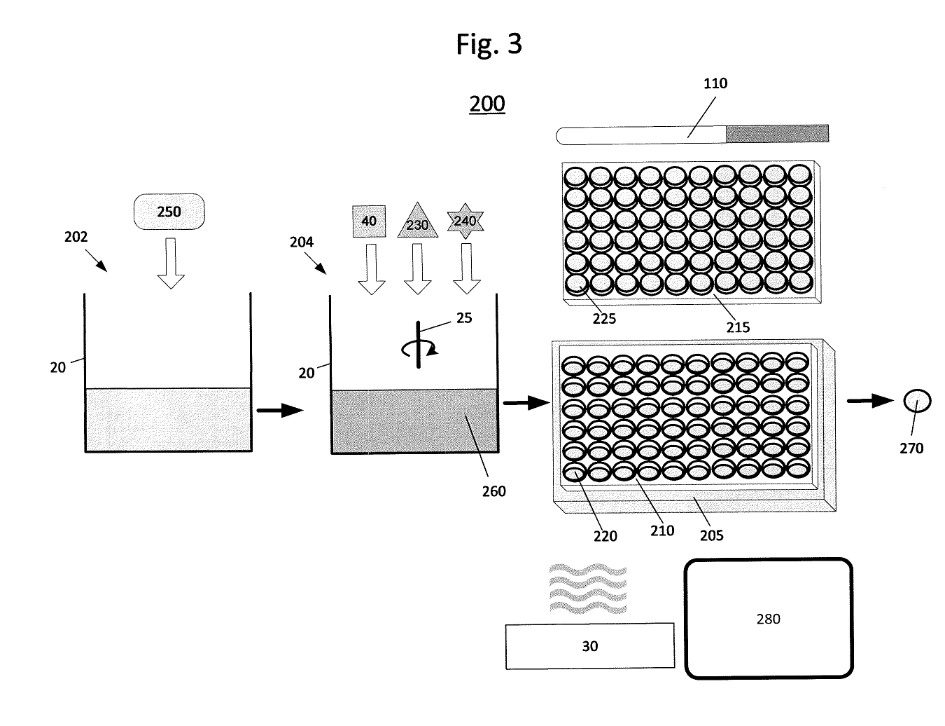

FIG. 3 illustrates an example system that can be utilized to produce a sublingual antidepressant and antianxiety tablet, arranged in accordance with at least some embodiments presented herein. Those components in FIG. 3 that are labeled identically to components of FIG. 1-2 will not be described again for the purposes of clarity.

System 200 may include chamber 20, mixing instrument 25, a base plate 205, a tablet mold base 210, and a tablet mold top 215. At 202, a sublingual tablet base 250 may be placed within chamber 20. Chamber 20 may be an aluminum chamber such as an anodized aluminum chamber. Sublingual tablet base 250 may be a rapid dissolve tablet base and may allow a sublingual antidepressant and antianxiety tablet comprising sublingual tablet base 250 to dissolve sublingually in a time range of 0-60 seconds.

At 204, mixing instrument 25 may be inserted into chamber 20 and ingredients 40, 230, and 240 may each be individually and respectively added and blended into sublingual tablet base 250. Mixing instrument 25 may be a manual mixing instrument such as a spoon or whisk, or an automated mixer.

Ingredient 40 may be in powder form. Ingredient 40 may include ketamine. Ingredient 40 may include ketamine hydrochloride (HCl) powder.

Ingredient 230 may be in powder or liquid form. Ingredient 230 may include a flavor. Ingredient 230 may make a sublingual antidepressant and antianxiety tablet comprising ingredient 230 more palatable. Ingredient 230 may enhance digestion and taste of a sublingual antidepressant and antianxiety tablet comprising ingredient 230. Ingredient 230 may be a raspberry flavor.

Ingredient 240 may be in powder or liquid form. Ingredient 240 may be a sweetener. Ingredient 240 may make a sublingual antidepressant and antianxiety tablet comprising ingredient 240 more palatable. Ingredient 240 may enhance digestion and taste of a sublingual antidepressant and antianxiety tablet comprising ingredient 240. Ingredient 240 may include a combination of vanilla and a sugar substitute such as STEVIA. Ingredient 240 may include about 61.5 weight percent vanilla and about 38.5 weight percent STEVIA.

As shown at 204, ingredient 40 may be added to chamber 20 and blended into sublingual tablet base 250. Ingredient 40 may be geometrically diluted into sublingual tablet base 250. Ingredient 40 may be mixed until ingredient 40 is evenly distributed throughout sublingual tablet base 250 as indicated by an even distribution of a color of ingredient 40 throughout sublingual tablet base 250.

As shown at 204, ingredient 230 may be added to chamber 20 and blended into sublingual tablet base 250. Ingredient 230 may be geometrically diluted into tablet base 250. Ingredient 230 may be mixed until ingredient 230 is evenly distributed throughout sublingual tablet base 250 as indicated by an even distribution of a color of ingredient 230 throughout sublingual tablet base 250.

As shown at 204, ingredient 240 may be added to chamber 20 and blended into sublingual tablet base 250. Ingredient 240 may be geometrically diluted into sublingual tablet base 250. Ingredient 240 may be mixed until ingredient 40 is evenly distributed throughout sublingual tablet base 250 as indicated by an even distribution of a color of ingredient 240 throughout sublingual tablet base 250.

A tablet mixture 260 may be formed by mixing ingredients 40, 230, and 240 into sublingual tablet base 250. Base plate 205 may be a glass plate or an ointment tile. Tablet mold base 210 may be positioned on top of base plate 205 so that a bottom side of tablet mold base 210 is abutted to base plate 205. Walls of tablet mold base 210 may define openings 220. Tablet mixture 260 may be placed or pressed into openings 220 defined by walls of tablet mold base 210. Tablet mold base 210 may be anodized aluminum, or some other non-permeable material, and may be configured to form uniform sized tablets. Walls of tablet mold base 210 may define 60 uniformly sized openings 220. Tablet mixture 260 may be pressed into openings 220 of tablet mold base 210 so as to completely fill openings 220. In some examples, spatula 110 may be used to press, level, and even out tablet mixture 260 into openings 220 of tablet mold base 210. In another example, tablet mold top 215 may be positioned on tablet mold base 210 such that tablet pegs 225 of tablet mold top 215 align with openings 220 defined by walls of tablet mold base 210. Tablet pegs 225 of tablet mold top 215 may press tablet mixture 260 into openings 220 defined by walls of tablet mold base 210.

A combination of tablet mold base 210, with openings 220 filled with tablet mixture 260, and tablet mold cover 215, may be heated to dry tablet mixture 260. Drying may be performed by placing the combination of tablet mold base 210 with openings 220 filled with tablet mixture 260 and tablet mold cover 215 by heater 30. Drying may be performed by placing tablet mold base 210 with openings 220 filled with tablet mixture 260 and tablet mold cover 215 in an oven 280. Heater 30 or oven 280 may heat tablet mold base 210 with openings 220 filled with tablet mixture 260 to a temperature of about 90 to 130 degrees Celsius for a time range of 10 to 15 minutes. Drying may be performed by allowing tablet mold base 210 with openings 220 filled with tablet mixture 260 and tablet mold cover 215 to dry at room temperature of 20 to 25 degrees Celsius to form tablet 270. After tablets 270 are dried within tablet mold base 210, pegs 225 of tablet mold top 215 may be used to push tablets 270 out of tablet mold base 210.

Tablet 270 may be a solid tablet with ingredients 40, 230, and 240 distributed evenly throughout tablet 270. Tablet 270 may include about 2.00 weight percent to about 23.00 weight percent of ingredient 40. Tablet 270 may include about 0.35 weight percent to about 0.65 weight percent of ingredient 230. Tablet 270 may include about 0.30 weight percent to about 0.50 weight percent of ingredient 240.

### Example 4

Tablet 270 may include:
2.5 weight percent of ingredient 40;
0.55 weight percent of ingredient 230; and
0.45 weight percent of ingredient 240.

### Example 5

A tablet mold base with 60 uniformly sized cavities may be utilized to mold 60 tablets 270 from tablet mixture 260 formed from the following quantities:
0.6 grams of ingredient 40. Ingredient 40 may be ketamine HCl powder;
0.064 grams of ingredient 230. Ingredient 230 may raspberry flavor;
0.052 grams of ingredient 240. Ingredient 240 may be a combination of vanilla and STEVIA;
11.22 grams of sublingual tablet base 250.

### Example 6

A tablet mold base with 60 uniformly sized cavities may be utilized to mold 60 tablets 270 from tablet mixture 260 formed from the following quantities:
1.50 grams of ingredient 40. Ingredient 40 may be ketamine HCl powder;
0.064 grams of ingredient 230. Ingredient 230 may raspberry flavor;
0.052 grams of ingredient 240. Ingredient 240 may be a combination of vanilla and STEVIA;
11.22 grams of sublingual tablet base 250.

### Example 7

A tablet mold base with 60 uniformly sized cavities may be utilized to mold 60 tablets 270 from tablet mixture 260 formed from the following quantities:
3.00 grams of ingredient 40. Ingredient 40 may be ketamine HCl powder;
0.064 grams of ingredient 230. Ingredient 230 may raspberry flavor;
0.052 grams of ingredient 240. Ingredient 240 may be a combination of vanilla and STEVIA;
11.22 grams of sublingual tablet base 250.

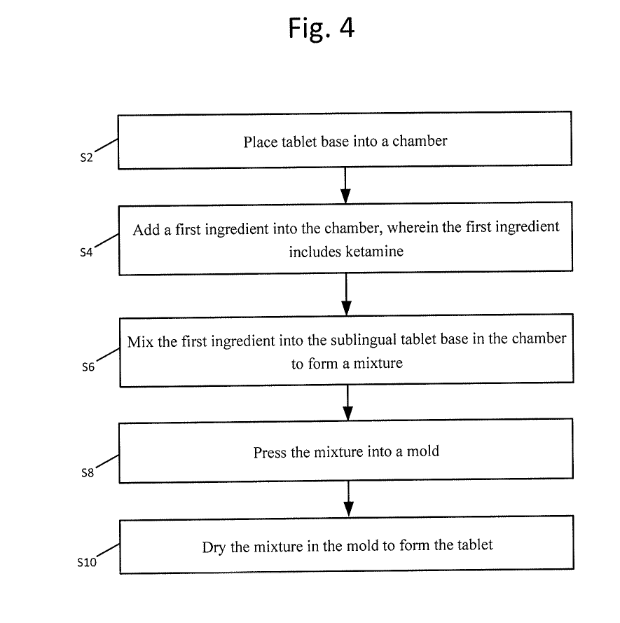

FIG. 4 illustrates a flow diagram of an example process to produce a sublingual antidepressant and antianxiety tablet 270. The process in FIG. 4 could be implemented using, for example, system 200 discussed above. An example process may include one or more operations, actions, or functions as illustrated by one or more of blocks S2, S4, S6, S8, and/or S10. Although illustrated as discrete blocks, various blocks may be divided into additional blocks, combined into fewer blocks, or eliminated, depending on the desired implementation.

Processing may begin at block S2, “Place sublingual tablet base into a chamber.” At block S2, a sublingual tablet base may be placed into a chamber. The sublingual tablet base may be a rapid dissolve tablet base and may allow a sublingual antidepressant and antianxiety table comprising the tablet base to dissolve sublingually in a time range of 0-60 seconds.

Processing may continue from block S2 to block S4, “Add a first ingredient into the chamber, wherein the first ingredient includes ketamine.” At block S4, a first ingredient may be added to the chamber. The first ingredient may include ketamine. The first ingredient may include ketamine hydrochloride (HCl) powder.

Processing may continue from block S4 to block S6, “Mix the first ingredient into the sublingual tablet base in the chamber to form a mixture.” At block S6, the first ingredient may be mixed into the sublingual tablet base in the chamber. The mixing may be performed by a manual mixing instrument such as a spoon or whisk, or an automated mixer. The first ingredient may be mixed until the first ingredient is evenly distributed throughout the tablet base as indicated by an even distribution of a color of the first ingredient throughout the tablet base.

Processing may continue from block S6 to block S8, “Press the mixture into a mold.” At block S8, the mixture may be pressed into a mold. The mold may be plastic, anodized aluminum, or some other non-permeable material. The mold may be configured with cavities to form uniform sized tablets. The mixture may be placed or pressed into the cavities of the mold. The mixture may be pressed into the cavities of the mold so as to completely fill the cavities of the mold. A scrapper or spatula may be used to press, level, and even out mixture in the cavities of the mold. In another example, a tablet mold top may be positioned on the mold such that tablet dimples of the tablet mold top align with the cavities of the mold base. The tablet dimples of the tablet mold top may press the tablet mixture into the cavities of the mold.

Processing may continue from block S8 to block S10, “Dry the mixture in the mold to form the tablet.” At block S10, the mixture in the mold may be dried to form the tablet. The mold with the cavities filled with the mixture may be heated to dry, such as by a heater, dried in an oven, or allowed to dry at room temperature of 90 to 130 degrees Celsius for a time range of 10 to 15 minutes.

A system in accordance with the present disclosure may be effective to produce a sublingual antidepressant and antianxiety lozenge or a sublingual antidepressant and antianxiety tablet. A potential benefit of the present application may be the treatment of depression effects in treatment-resistant depression. An embodiment of the present application may provide a more rapid effect than antidepressant treatments which include therapies that target monoaminergic (MA) systems and require 4-6 weeks of administration to achieve effects. An embodiment of the present application may provide a higher efficacy rate than therapies that target monoaminergic (MA) systems. An embodiment of the present application may provide a treatment for anxiety including anxiety related to post traumatic stress disorder and anxiety related to addiction. An embodiment of the present application may provide a treatment for depression and anxiety including depression and anxiety related to postpartum depression, bipolar disorder, Alzheimer's disease and dementia, and other metal health disorders.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A sublingual antidepressant and antianxiety tablet comprising a sublingual tablet base and 2.00 weight percent to 25.00 weight percent ketamine.

2. The tablet of claim 1, wherein the sublingual tablet base is a rapid dissolve tablet base and allows a sublingual antidepressant and antianxiety table comprising the sublingual tablet base to dissolve sublingually in a time range of 0-60 seconds.

3. The tablet of claim 1, further comprising 0.35 weight percent to 0.65 weight percent of a flavor.

4. The tablet of claim 1, further comprising 0.30 weight percent to 0.05 weight percent of a sweetener.

5. The tablet of claim 1, comprising:
2.00 weight percent to 3.00 weight percent ketamine.

6. The tablet of claim 1, comprising:
4.60 weight percent to 5.40 weight percent ketamine.

7. The tablet of claim 1, comprising:
11.20 weight percent to 12.00 weight percent ketamine.

8. The tablet of claim 1, comprising:
19.00 weight percent to 23.00 weight percent ketamine.

9. The tablet of claim 1, wherein the ketamine includes ketamine hydrochloride powder.

10. A method to produce a sublingual antidepressant and antianxiety tablet, the method comprising:
placing sublingual tablet base into a chamber;
adding a first ingredient into the chamber, wherein the first ingredient includes ketamine;
mixing the first ingredient into the sublingual tablet base in the chamber to form a mixture;
pressing the mixture into a mold; and
drying the mixture in the mold to form the sublingual antidepressant and antianxiety tablet.

11. The method of claim 10, further comprising, prior to pressing the mixture into the mold:
adding a second ingredient to the chamber, wherein the second ingredient includes a flavor; and
mixing the second ingredient with the mixture in the chamber.

12. The method of claim 11, wherein the flavor is raspberry flavor.

13. The method of claim 10, further comprising, prior to pressing the mixture into the mold:
adding a second ingredient to the chamber, wherein the second ingredient includes a sweetener; and
mixing the second ingredient with the mixture in the chamber.

14. The method of claim 13, wherein the sweetener includes vanilla and a sugar substitute.

15. The method of claim 10, further comprising, prior to pressing the mixture into the mold:
adding a second ingredient to the chamber, wherein the second ingredient includes a flavor;
mixing the second ingredient with the mixture in the chamber;
adding a third ingredient to the chamber, wherein the third ingredient includes a sweetener; and
mixing the third ingredient with the mixture in the chamber.

16. The method of claim 13, wherein the flavor is raspberry flavor and the sweetener includes vanilla and a sugar substitute.

17. The method of claim 16, wherein:
the mold includes sixty uniformly sized cavities;
the sublingual tablet base weighs 11.224 grams;
the ketamine weighs 0.300 to 3.00 grams;
the raspberry flavor weighs 0.064 grams; and
the vanilla and sugar substitute weighs 0.052 grams.

18. The method of claim 10, wherein the ketamine includes ketamine hydrochloride powder.

19. A sublingual antidepressant tablet comprising sublingual tablet base and 0.005 to 0.050 grams of ketamine.

20. The tablet of claim 19, wherein the ketamine includes ketamine hydrochloride powder.
